(19) United States
(12) Patent Application Publication
(10) Pub. No.: US 2016/0320373 A1
(43) Pub. Date: Nov. 3, 2016

(54) Re-Usable Analyte Detector and Methods

(71) Applicant: BOARD OF TRUSTEES OF THE LELAND STANFORD JUNIOR UNIVERSITY, Palo Alto, CA (US)

(72) Inventors: James S. HARRIS, Jr., Stanford, CA (US); Ronald W. DAVIS, Palo Alto, CA (US); Rahim ESFANDYARPOUR, Stanford, CA (US)

(21) Appl. No.: 15/108,559

(22) PCT Filed: Jan. 5, 2015
(86) PCT No.: PCT/US2015/010161

§ 371 Date: Jun. 27, 2016

Related U.S. Application Data
(60) Provisional application No. 61/930,196, filed on Jan. 22, 2014.

(51) Int. Cl. G01N 33/543; C12Q 1/68; B01L 3/00

(52) U.S. Cl. CPC G01N 33/5438; G01N 33/54326; B01L 3/502715; B01L 3/502761; C12Q 1/6869; B01L 2300/0887; B01L 2300/0645; B01L 2300/12; B01L 2300/16

(57) ABSTRACT

Devices and methods for detecting the presence of one or more analytes, such as biomolecules (FIG. 2). An analyte detection device includes at least two conductive structures disposed between three insulating structures and a gap exists in the structures that defines a channel such that at least four electrodes capable of measuring an electrical property are present in the channel. Methods for improving analyte detector performance through low-salt buffer washes also are disclosed.

### CROSS REFERENCE

This application claims priority to U.S. provisional patent application 61/930,196 filed on Jan. 22, 2014, which is incorporated by reference herein in its entirety.

### STATEMENT REGARDING FEDERALLY SPONSORED RESEARCH OR DEVELOPMENT

This invention was made with government support under HG000205 awarded by The National Institutes of Health. The U.S. government has certain rights in the invention.

### BACKGROUND OF THE INVENTION

Detection of analytes, such as biomolecules, has various applications in medicine, biotechnology, our understanding of biology, and “personalized medicine.” The biomolecules of interest may range from proteins and nucleic acids to whole cells and metabolites.

One class of analyte detectors is electrical biosensors, which show promise for point-of-care and other applications. Many affinity-based electrical biosensors are designed to detect or quantify a biochemical molecule, such as a particular DNA sequence, a particular protein or cells. The main requirements for a biosensor are selectivity and sensitivity.

Selectivity can be referred to as affinity-based sensing, which means that the sensors use an immobilized capture probe that binds the molecule being sensed (the target or analyte) selectively. These sensors transfer the challenge of detecting a target in solution into the detection of a change at a localized surface, which can be measured in a variety of ways such as measurement of currents and/or voltages, change of capacitance, resistance, conductance, relative dielectric permittivity or impedance.

Possibly the main advantage of some of the electrical biosensors is their ability to perform label-free detection. Most other types of biosensors require having a label attached to the target biomolecule or to a secondary protein. For these types of biosensors the assumption is that the amount of detected labeled-biomolecules corresponds to the number of bound targets. There are varieties of labels, such as those containing fluorophores, magnetic beads, and active enzymes with a detectable product, allowing facile target conjugation and convenient detection.

However, labeling a biomolecule could considerably change its binding properties, which is especially problematic for protein targets. Further, labeling requires extra time, expense, and sample handling. In contrast, label-free operation has the advantage of detection of target-probe binding in real time (which is generally not possible with label-based systems), and requires less time and expense due to omitting the labeling step. Finally, the salt conditions used for many electrical biosensors can cause problems with target binding and detection.

Therefore, there is a need for analyte detectors, such as electrical biosensors, that can perform label-free detection and further for detectors and methods that provide both selectivity and increased sensitivity as compared to conventional devices and methods of analyte detection.

### SUMMARY OF THE INVENTION

Embodiments described herein include an analyte detection device featuring at least two conductive structures disposed between three insulating structures such that the conductive structures are insulated from each other and an external environment by the insulating structures. The device further includes a gap defining a channel within the conductive and insulating structures such that at least four electrodes capable of measuring an electrical property are present in the channel. Preferably, the gap is symmetrically disposed in the conductive and insulating structures as shown in the figures.

Additional embodiments are directed to methods of detection involving the use of low salt buffer washes.

Among the new and inventive aspects, devices and methods described herein can be multiplexed in an array, are sensitive, re-usable, relatively inexpensive and can produce fast, real-time detection results.

These and other aspects of the invention will be apparent upon reference to the following detailed description and figures. To that end, any patent and other documents cited herein are hereby incorporated by reference in their entirety.

### BRIEF DESCRIPTION OF THE DRAWINGS

FIG. 1 illustrates detection of Vascular Endothelial Growth Factor (VEGF) for cancer diagnosis with a control experiment using physiological salt buffer for sample preparation. Anti-VEGF was immobilized on the ODTS-Silanized surface of the sensors overnight. VEGF with the concentration of 50 ng/ml was detected. Results indicate a 6% decrease in impedance level after VEGF binding with no change after 40 μg/ml of Streptavidin was injected to the channel indicating no binding to Anti-VEGF (as shown in Inset).

FIG. 2 depicts a schematic representation of an embodiment of an analyte detection device of the invention.

FIG. 3 depicts a further application of the device in FIG. 2.

FIG. 4 illustrates a multiplex embodiment of the device in FIG. 2. The change of measured impedance is due to capturing of biomolecules. Detectable concentration can be identified based on the percentage of change.

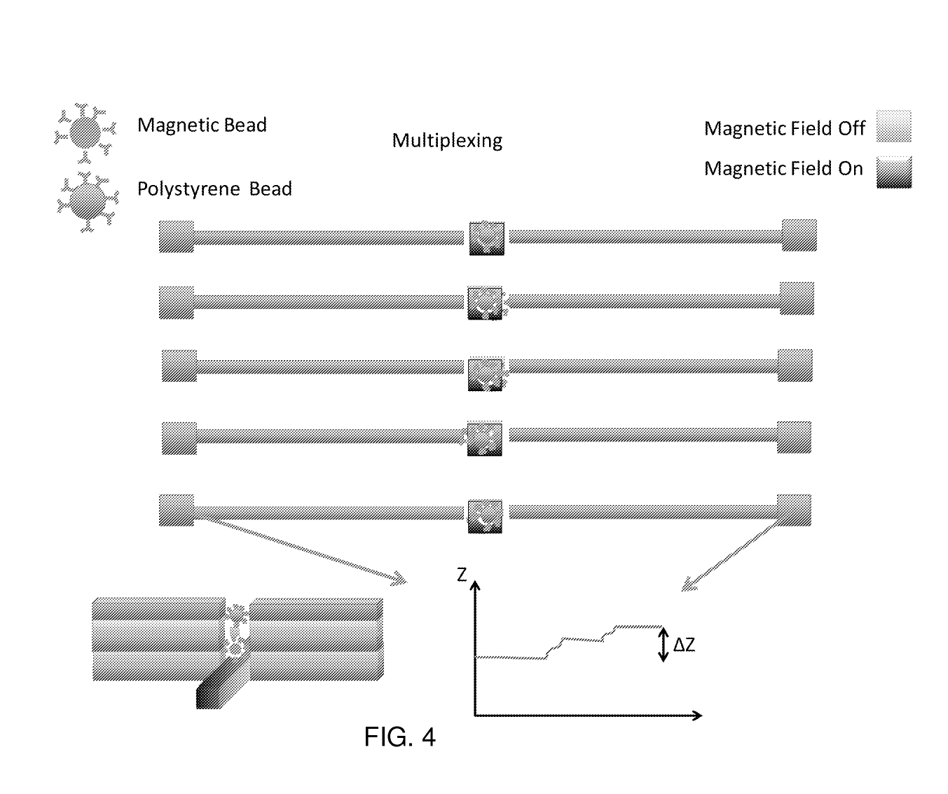

FIG. 5 depicts two configurations of a magnetic field source of a re-usable magneto-trapping array embodiment of the invention which can be switched on/off (permanent magnetic field source). The magnetic field source can include an on-chip integrated inductor inside, which can induce a magnetic field by applying voltage/current to it.

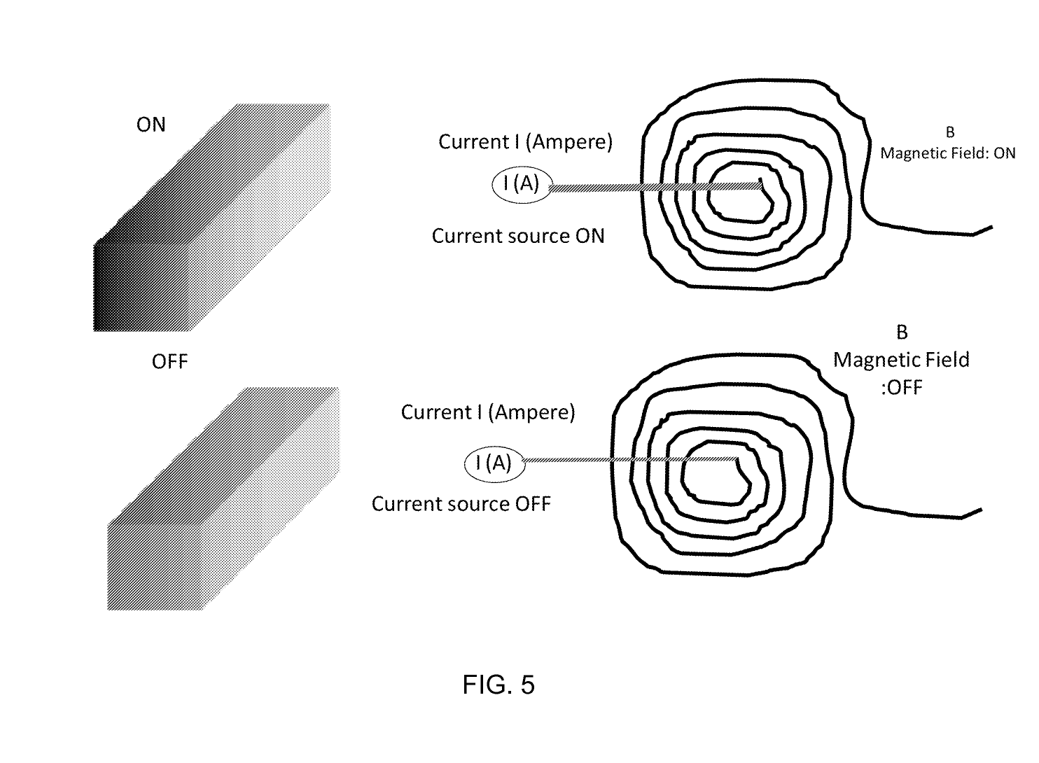

### DETAILED DESCRIPTION OF THE INVENTION

In one aspect, an improved method for electrical analyte detection (i.e., utilizing an analyte detector that measures changes in one or more electrical properties) is disclosed. In a further aspect, such methods can be performed with biosensors.

Some biosensors can provide limited sensitivity, working well with biomolecules only in low salt solutions. But low salt concentration buffers can affect the functionality of the biomolecules specially proteins and cells. Methods described herein address this issue. The methods can be used for the detection of substantially all biomarkers such as cells, DNAs, proteins, bacteria, viruses or any other type of biomolecules or any type of particles with many types of electrical biosensors. These methods can also be used for the detection of viruses or bacteria by using their appropriate receptors. Further, these methods can be used for substantially all the label-free biosensors as well as those types of biosensors that require having a label attached to the target proteins or secondary proteins.

Currently, many electrical biosensors (that can do real time electrical property measurement) perform the detection in two steps. First, capture probes are loaded to the sensory part of the sensor while they measure the property of interest (impedance, current, voltage, conductance, capacitance, permittivity etc.). Next, the target biomolecules are introduced to the sensor. A specific binding occurs between the capture probes and the target biomolecule, which results in a change in the measured signal (impedance, current, voltage, conductance, capacitance, permittivity etc.).

The difference between the measured signal after the introducing and binding of these capture probes to the sensor step and the measured signal after introducing the target biomolecules and binding of them to the receptors step constitutes a detection signal that is due to the specific binding.

Biomolecules need to be in a physiological salt buffer solution to have their functionality. In other words, they may loose their functionality and their binding properties if they are diluted in lower than physiological concentration salt buffer or kept in a lower salt environment for a long time. Indeed, the detection steps for some biosensors involve a wash step using a high salt buffer to dilute the biomolecules of interest. The difference of the measured signal after each wash step is the presumed detection signal. However, having a large number of ions in a physiological or higher concentration salt buffer has caused many biosensors to suffer from the ionic charge accumulation at the sensory part of the sensor (which is modeled as double layer capacitance). This layer of charge can decrease their sensitivity or affect the functionality of the sensor.

Accordingly, in one embodiment, a sensing material, such as capture probes (e.g., receptor proteins), is diluted in a physiological (>100 mM) salt buffer and left to bond to the sensory part of the electrical sensor. Next, the sensor is washed with a less than physiological concentration salt buffer (i.e., under 100 mM) and an electrical property (such as current, voltage, resistance, capacitance, impedance etc.) is measured to find the base line while the bioreceptors are bonded to the sensory part of the sensor. Then a sample that may contain target biomolecules of interest is diluted in a physiological salt buffer and introduced to the sensor.

The target biomolecules are free to bond to the capture probes (bioreceptors) present at the sensory part of the sensor without losing their functionality. The sensor is washed again with a less than physiological concentration salt buffer. Then a second measurement of an electrical property is completed. The difference between two measurements after the two wash steps is the detection signal for the analyte/target of interest.

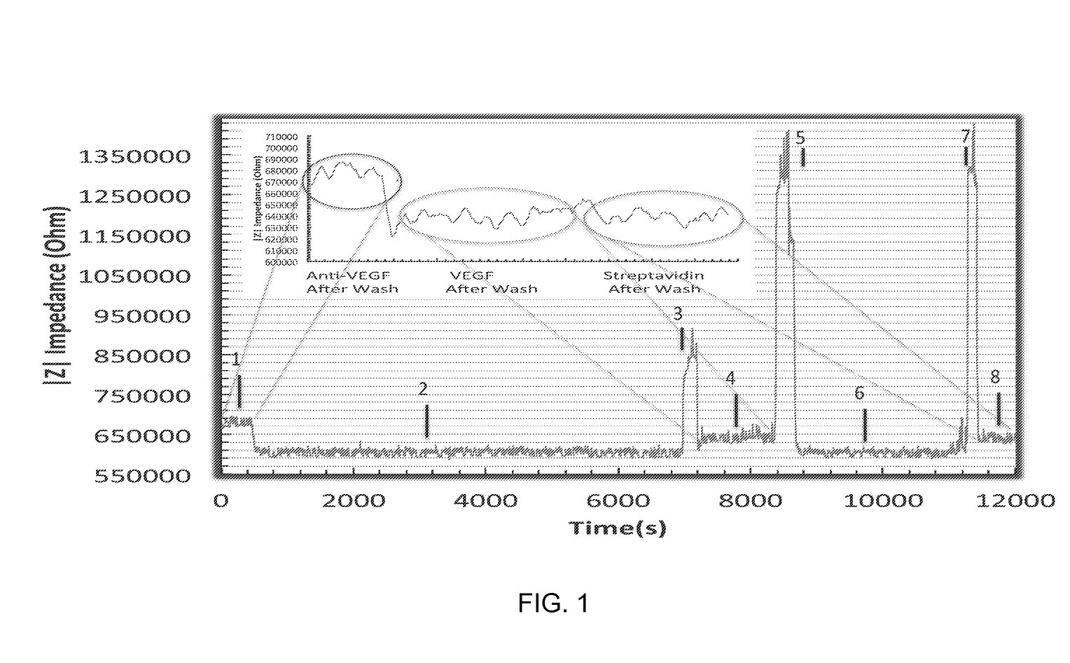

In a specific example, representative data for which is shown in FIG. 1, detection of a clinically relevant cancer biomarker (vascular endothelial growth factor (VEGF)) suspended in physiological salt buffer (>100 mM) with a detection limit of 50 ng/ml has been demonstrated. VEGF was diluted in 1× PBS (Phosphate Buffer Saline with 114 mM NaCl), which has the physiological salt concentration (>100 mM). The wash buffer in this example is lower salt concentration buffer (˜1 μM phosphate buffer solution containing 2 μM NaCl with pH 7.4) solution.

There are at least two advantages of using the improved methods. The first one is that by completing the first washing step, all non-target, non-specific and unbonded biomolecules are washed out so that they do not affect the measured signal. The second advantage is that the second wash step removes the accumulated ions at the sensory part of the sensor. Thus, no ions accumulate on the sensory part of the sensor and affect or distort the electrical measurements (and create another time dependent layer of charge). Therefore, the change of measured electrical property (e.g., impedance, current, voltage, conductance, capacitance, permittivity etc.) is substantially due to the binding of the target biomolecules (with their own charges) to the sensing material, which changes the relative dielectric permittivity and conductivity of that region of the buffer (i.e., the interface of buffer and the sensory part of the senor).

As mentioned above, these changes can appear and be measured as a change of the measured current, voltage, capacitance, resistance, conductance, permittivity, impedance or other electrical properties. Thus, using this method with different types of electrical biosensors results in a matrix insensitive property for them, which improves their sensitivity and their detection limit for the electrical detection of different types of targets such as biomarkers. Thus, this method can substantially push the limits of biosensors in, for example, medical applications.

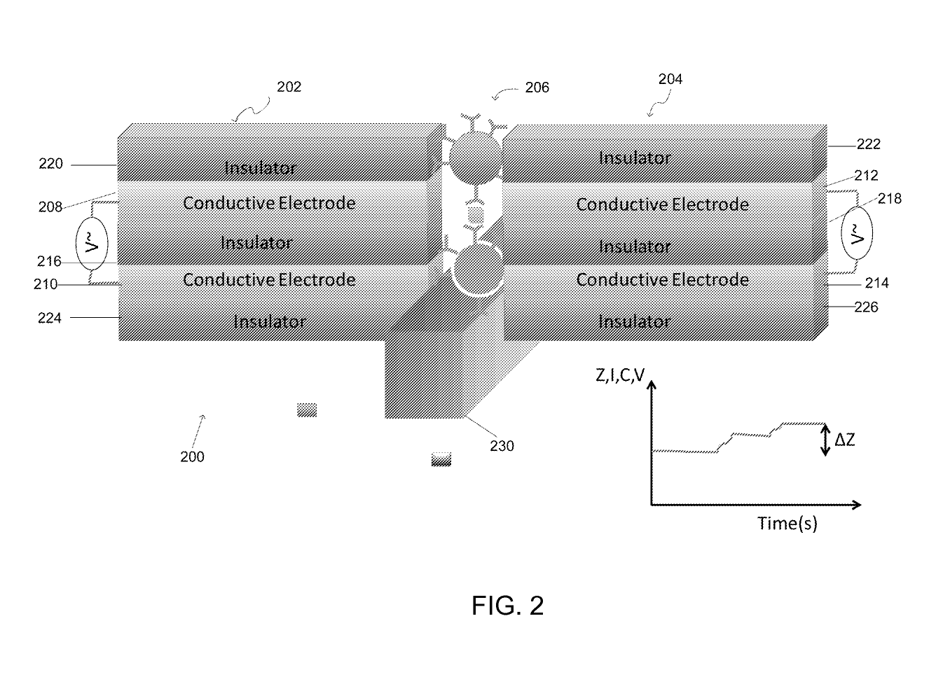

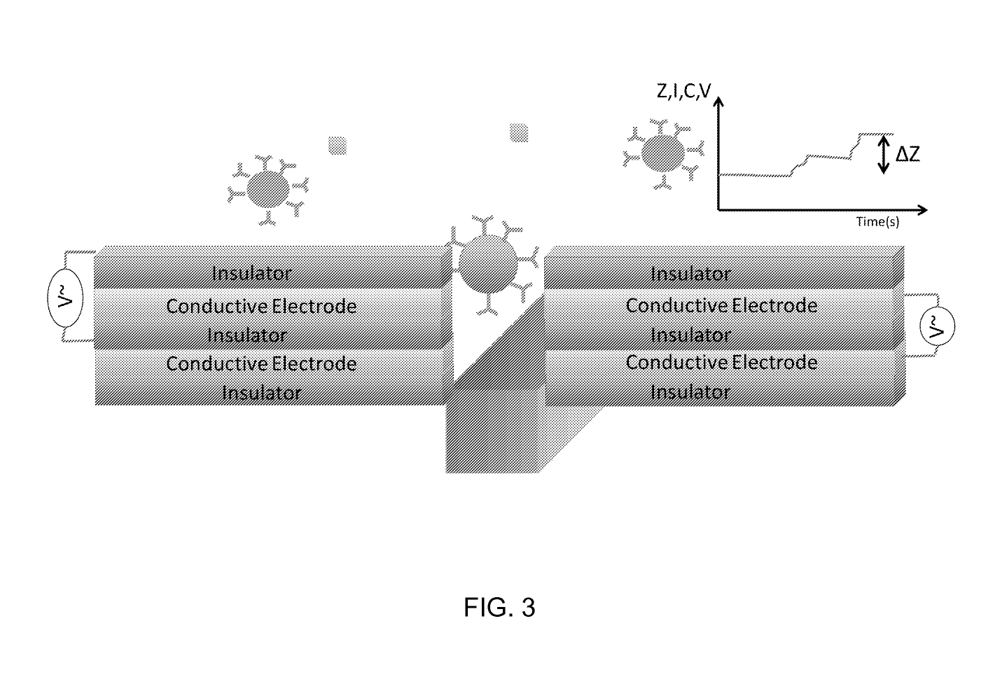

In another embodiment, a highly sensitive, real time, cost-effective and re-useable analyte detector comprising a biosensor is disclose (see FIGS. 2 and 3). As schematically shown in FIG. 2, sensor 200 includes two bar shape structures 202 and 204 in each side of a channel 206. Each bar 202 and 204 has two conductive layers 208 and 210, and 212 and 214, respectively, with insulation layers 216 and 218 in between. There are also two other protective (insulation) layers on top (220 and 222) and underneath of the bars, protective layers 224 and 226.

Each conductive and insulating layer is configured to define at least four operable (i.e., any other components needed for the detection/measurement of electrical properties are included) electrodes (208, 210, 212, and 214) in the channel 206. In most applications, the channel 206 will comprise a microfluidics channel.

Each conductive layer measures the passing current (can be modeled as voltage, impedance or other electrical properties) through the other electrodes continuously. Having four electrodes has been discovered to increase the sensitivity of the sensor and accuracy of the measured signals. However, more than four electrodes in each channel may suffice. The gap or channel region between the structures defines the sensory part of the sensor.

In an embodiment, there is a locally but externally introduced magnetic field source 230 substantially in the sensory part of the sensor (this magnetic field can be introduced by a locally fabricated inductor at a bottom of the sensory part of the sensor or with an external circuitry board or any other source of magnetic field). Since this magnetic field is applied substantially externally, its magnitude can be adjusted or it can be turned on or off.

FIG. 2 depicts a schematic illustration of biomolecules or particles (e.g. protein, cell, bacteria, virus etc.) detection system for a blood sample utilizing a re-usable “magneto-trapping array,” according to a further embodiment. In this embodiment, the steps for operation include, for example:

1) Magnetic field is off.

2) Turn on magnetic field resulting in the magnetic particles or beads covered with appropriate capture probes adsorbing to the magnetic bar (magnetic field source).

3) Inject blood sample with target biomolecules (e.g. antigen, cancer cell, bacteria etc.) into the channel, target biomolecule will bind to the capture probes located on the surface of magnetic beads

4) To amplify the measured signal, secondary particle such as polystyrene beads with the same capture probes are injected and bound to the target biomolecules.

5) Wash away all non-bound or non-desired species.

6) Turn off magnetic field; wash the system to re-use the sensor.

FIG. 3 also depicts a schematic illustration of a blood purification and/or cancer cell detection system using a re-usable magneto-trapping array. In this embodiment, steps for operation include, for example:

1) Magnetic field is off.

2) Specific capturing probes covered magnetic beads (or particles) result in specific binding between a cancer cell or any other biomolecule of interest in the channel.

3) Turn on magnetic field which causes the dipole of the magnetic bead/cell (or proteins, virus etc.) to adsorb to the magnetic bar.

4) To amplify the measured signal, secondary bead or particle (e.g. polystyrene beads) with the same capturing probes are injected and bound to the target biomolecules.

5) Wash away all non-bound or non-desired species.

6) Turn off magnetic field; wash the system to re-use the sensor.

Sequencing or detection can be accomplished using the magnetic trapping array embodiments as follows. First, while the magnetic field is off, magnetic beads covered with arbitrary biomolecules (e.g. antibodies or DNAs) are injected into the channel. By turning the magnetic field on, those capturing probes covered magnetic beads are attracted to the sensitive part of each sensor and are trapped there. A base line signal is then detected and measured following immobilization of the biomolecule covered magnetic beads.

Following this immobilization step, a sample containing the biomolecule of interest (such as antigen, DNA bases, bacteria or viruses but not limited to) is injected into the channel. Binding of the biomolecules of interest to the capturing probes covered magnetic beads results in a change in a measured electrical properties such as current (impedance, voltage, permittivity, etc.). This change can be due to the change of presented charges, ionic current (or pH in case of DNA sequencing), relative dielectric permittivity (RDP) or the other electrical properties of the sensory part of the sensor.

Next a washing step was completed to remove, non-bound biomolecules. To increase amplification of the measured signal (for the protein detection, or cell, virus and bacteria capturing or the DNA sequencing), secondary beads are injected into the channel. These secondary beads may include polystyrene beads (covered with the same capturing probes as the first injected magnetic beads), which also bond to the previously injected target biomolecules. A second washing step is completed to remove non-bound secondary beads from the channel. A final measurement of the current is detected and recorded.

When the experiment above is finished, the external magnetic field is turned off Since there is no further magnetic force, a simple washing step removes all of the biomolecules and beads to provide for cleaning of the sensors as before the experiment. Each sensor can be re-used many times without any damage or need for a replacement.

By substantially applying the magnetic field to the micro (or nano) gap size sensory part of the sensor, at least a single bead can be captured. As a result, a real time and very fast sequencing or detection can be completed.

In another embodiment, the trapping array can also be used for a fast, simple, accurate, high volume, and non-expensive blood purification method by adjusting the magnitude of the magnetic field at the final step. By applying a weaker magnetic field at the final step (after secondary beads wash step), the chain multiplex structures of polystyrene beads-(cell, virus, bacteria or any desired proteins)-magnetic beads can be released and separated out from the free magnetic beads by a washing buffer.

Since there is a fluidic force applied to the secondary beads (e.g. polystyrene beads), their attachment to the weak magnetic field will yield. As a result, they can be detached from the sensory part of the sensor and then be transported to a separated second chamber. Free magnetic beads (not having middle biomolecules and secondary beads), will remain strongly attached to the surface of the sensor.

In an embodiment, the reusable magnetic array may be used for influenza virus detection at a location such as an airport, which can spread such viruses quickly. Currently the best existing method takes around 4 hours for detection. However, using the sensors and methods of this invention, the influenza virus detection could take less than 20 minutes.

The following claims are not intended to be limited to the materials and methods, embodiments, and examples described herein.

1. An analyte detection device, comprising:
at least two conductive structures disposed between three insulating structures such that said conductive structures are insulated from each other and an external environment by said insulating structures; and
a gap defining a channel formed within said conductive and insulating structures such that at least four electrodes capable of measuring an electrical property are present in said channel.

2. The detection device of claim 1, further including a magnetic field source disposed at a bottom of said channel.

3. The detection device of claim 1, further including a sensing substance disposed within said channel.

4. The detection device of claim 2, wherein a plurality of biomolecules-coated particles are disposed within said channel.

5. The detection device of claim 1, wherein said conductive and insulating structures comprise alternating layers of insulating and conductive material.

6. The detection device of claim 1, further comprising a plurality of biomolecules-coated secondary particles disposed within said channel.

7. A method for analyte detection through changes in an electrical property, comprising the steps of:
measuring an electrical property before and after an analyte is added to a channel defined by at least four electrodes, said at least four electrodes being further defined as a gap in at least two conductive structures disposed between three insulating structures.

8. A method for detecting an analyte through changes in an electrical property, comprising:
washing an electrical analyte detector sensitized with a material capable of binding a target of interest with a less than physiological concentration salt wash,
measuring an electrical property,
introducing a sample to said analyte detector,
washing said electrical analyte detector with a less than physiological concentration salt wash; and
measuring said electrical property again.

9. The method of claim 8, wherein said analyte detector comprises at least two conductive structures disposed between three insulating structures such that said conductive structures are insulated from each other and an external environment by said insulating structures, and a gap defining a channel is formed within said conductive and insulating structures such that at least four electrodes capable of measuring said electrical property are present in said channel.

10. The method of claim 8, wherein said less than physiological concentration salt wash buffer is less than 100 mM.

11. The method of claim 8, wherein said less than physiological concentration salt buffer is about 1 μM.
